(19) United States
(12) Patent Application Publication
(10) Pub. No.: US 2011/0212040 A1
(43) Pub. Date: Sep. 1, 2011

(54) STABILIZED SUNSCREEN COMPOSITIONS

(76) Inventors: Lea VON OPPEN BEZALEL, Berlin (DE); Fabien HAVAS, Rehovot (IL); Ilana KACHKO-CHERNETSKY, Beer Sheba (IL); Alex ALILUIKO, Rehovot (IL); Danit FISHBEIN MANOR, Moshav Beer Tuvia (IL); Olga BEN CHITRIT, Ashdod (IL); Inon PERRY, Tel Aviv (IL)

(21) Appl. No.: 13/035,976

(22) Filed: Feb. 27, 2011

Related U.S. Application Data
(60) Provisional application No. 61/308,976, filed on Feb. 28, 2010.

(51) Int. Cl. A61K 8/31; A61K 8/368; A61Q 17/04

(52) U.S. Cl. 424/60; 424/59

(57) ABSTRACT

Sunscreen compositions comprising at least one sunscreen active wherein said sunscreen active and composition are stabilized by the addition natural or synthetic phytoene or phytofluene or mixtures thereof and optionally at least one other natural or synthetic carotenoid. A method of stabilization of sunscreen compositions and sunscreen actives, especially avobenzone, when exposed to UV light and free radicals attack.

### CROSS-REFERENCE TO RELATED APPLICATIONS

The present application claims priority to U.S. Provisional Patent Application No. 61/308,976 filed Feb. 28, 2010, which is incorporated herein by reference in its entirety.

### BACKGROUND OF THE INVENTION

The present invention relates to the field of sunscreens and other products meant for skin protection against UV radiation.

Sunscreens are topical compositions which are applied to the skin of a person in need thereof, in order to obtain protection from undesirable ultraviolet (UV) radiation in the sunlight. The UV radiations of interest for sunscreens are UVA (long wave UV or black light), UVB (medium or short to medium wave UV) and UVC (short wave UV). The efficiency of the sunscreens is measured by a laboratory measure of effectiveness named Sun Protection Factor (SPF) for the protection against UVB radiation and UVA Protection Factor (UVA-PF) for the protection against UVA radiation. The higher the SPF, the more protection a sunscreen offers. A number of sunscreen actives (also known as UV-filters) are approved for use in various countries. UVC protection is currently not evaluated by standardized measures as it is not considered to be derived from exposure to the sun; however it is of considerable risk indoors.

The prominent sunscreen actives are p-aminobenzoic acid, Pamidate O, phenylbenzimidazole sulfonic acid, cinoxate, dioxybenzone, oxybenzone, octyl methoxy cinnamate, octyl salicylate, sulisobenzone, avobenzone. Two of the inorganic sunscreen actives used in sunscreen compositions are zinc oxide (ZnO) and titanium oxide (TiO₂).

### SUMMARY OF THE INVENTION

The present invention successfully addresses unmet needs, providing innovative sunscreen and UVC protection compositions comprising at least one sunscreen active with enhanced stability of the sunscreen actives towards sunlight, free radical attack or combinations thereof. In addition, the new, stabilized sunscreens provide improved safety, at reasonable costs. The enhanced stability is preserved also in the presence of inorganic sunscreens like ZnO and TiO2.

The enhanced stability of the sunscreen and UVC protection compositions is achieved by addition to the sunscreen compositions of natural or synthetic phytoene, phytofluene or mixtures thereof (P&P) and optionally of additional natural or synthetic carotenoids.

One of the prominent sunscreen actives is avobenzone, which is an effective and broadly used UV-filter. Avobenzone is susceptible to degradation under the influence of sunlight (especially UV light) and even more so in the presence of ZnO and TiO₂.

The instability of sunscreen actives and especially the instability of avobenzone.to sunlight, free radical attack or combinations thereof, especially in the presence of ZnO and TiO₂ causes degradation of the sunscreen compositions and decreased stability and effectiveness.

Avobenzone is an oil soluble broad spectrum sunscreen active with an absorption maximum of 357 nm (6). The INCI name of avobenzone is butyl methoxydibenzoylmethane.

Sunscreen actives and especially avobenzone are degraded by UV light and normal day light. Therefore the sunscreen content of the composition will be drastically reduced over time during the course of normal use and thereby the capability to protect against UV radiation. Furthermore, any such degradation will lead to the formation of free radicals and subsequent oxidative stress with ensuing tissue damage. Addition of carotenoids, especially colorless carotenoids may diminish this course of events, as can be seen from the Examples.

Controlled degradation studies have shown that 1 hour of sunlight exposure reduces avobenzone absorbance by 36% (7-10, 16). We have also shown that 10 hrs exposure to UVA+UVB+UVC light is conducive to 45% avobenzone degradation (Example No. 4) and that avobenzone undergoes substantial degradation under exposure to UV light in a variety of conditions (Examples No. 2, 3, 4).

Avobenzone degrades faster when exposed to light in combination with inorganic sunscreens like zinc oxide (ZnO) and titanium dioxide (TiO₂). These compounds protect against UV irradiation by reflection of the light; however, this action also generates free radicals that can additionally degrade the avobenzone (18).

Free radicals generation by ZnO and TiO₂ when exposed to UV is known to decrease the stability of other components in the sunscreen compositions as well as increasing the risk of oxidative damage from the free radicals generated (17-19).

Avobenzone, when formulated alone or in combination with the inorganic sunscreens is significantly unstable and easily decomposed when exposed to UV

Though avobenzone is in use for a long time, and is an efficient and relatively cheap sunscreen with low irritancy, no efficient method has been found for its stabilization, as is shown by continuing attempts of the industry to find new stabilization methods. Some of the stabilization methods proposed, like microencapsulation or combinations with other sunscreens, provide some stabilization, but add additional, potentially toxic materials to the compositions.

There is a long felt need for a safe and effective stabilization method for this important sunscreen agent.

Stabilization of avobenzone by free radical quenching and photo stabilization is therefore needed to maintain the optimum protective activity from this sunscreen.

A number of stabilizers of avobenzone have been claimed (11-15). However, these stabilizers, aside from their toxicity risks, do not provide a satisfactory solution to the formulatory challenges, as shown by the continuing research in this field.

It was surprisingly found that natural or synthetic colorless carotenoids, phytoene, phytofluene or mixtures thereof demonstrate excellent efficacy in the stabilization of sunscreens, especially avobenzone, filling a long-felt need in the sunscreen industry. These unique and proprietary colorless carotenoids absorb light at the UVB, UVA range respectively (1-5) The colorless carotenoids have the ability to both absorb UV light and quench UV generated free radicals such as hydroxyl radicals.

These and other aspects of the invention will become apparent from the description of the invention, which follows below.

### BRIEF DESCRIPTION OF THE DRAWINGS

FIGS. 1a-d depict the degradation of avobenzone in various solvents: avobenzone remaining in hydrogenated polydecene (NB), squalane (SQ), and colorless jojoba (JJ) oils under UV irradiation for different periods of time.

FIG. 1a Degradation of avobenzone in various solvents: avobenzone remaining in hydrogenated polydecene (NB), squalane, and colorless jojoba oils under UVA irradiation vs. time (18 hrs).

FIG. 1b Degradation of avobenzone in various solvents: avobenzone remaining in hydrogenated polydecene (NB), squalane (SQ), and colorless jojoba (JJ) oils under UVA irradiation vs. time (5 hrs).

FIG. 1c—Degradation of avobenzone in various solvents: avobenzone remaining in hydrogenated polydecene (NB), squalane (SQ), and colorless jojoba (JJ) oils after 18 hours exposure to UVA irradiation.

FIG. 1d—Degradation of avobenzone in various solvents: avobenzone remaining in hydrogenated polydecene (NB), squalane (SQ), and colorless jojoba (JJ) oils after 5 hours exposure to UVC irradiation.

FIGS. 2a-c—depict the degradation of avobenzone under single UV domain irradiation

FIG. 2a—Percentage of initial avobenzone remaining in compositions of increasing concentrations of phytoene and phytofluene after 18 hours of UVA exposure. Phytoene and phytofluene mixture is represented by the exemplary stabilizing composition IBR-TCLC® Concentrated in Squalane 0707.

FIG. 2b—Percentage of initial avobenzone remaining in the presence of increasing concentrations of phytoene and phytofluene after 18 hours of UVB exposure. Phytoene and phytofluene mixture is represented by the exemplary stabilizing composition IBR-TCLC® Concentrated in Squalane 0707.

FIG. 2c—Percentage of initial avobenzone remaining in the presence of increasing concentrations of phytoene and phytofluene after 5 hours of UVC exposure. Phytoene and phytofluene mixture is represented by the exemplary stabilizing composition IBR-TCLC® Concentrated in Squalane 0707.

FIGS. 3a-b—depict the degradation of avobenzone under combined UV domains' irradiation

FIG. 3a—Percent of initial avobenzone remaining in the presence of increasing concentrations of phytoene and phytofluene after 18 hours of combined UVA+B exposure. Illuminants combined at 4:2 ratios; phytoene and phytofluene mixture is represented by the exemplary stabilizing composition. IBR-TCLC® Concentrated in Squalane 0707.

FIG. 3b—Percent of initial remaining in the presence of increasing concentrations of phytoene and phytofluene after 10 hours of combined UVA+B+C exposure. Illuminants combined at 3:2:1 ratios; phytoene and phytofluene mixture is represented by the exemplary stabilizing composition. IBR-TCLC® Concentrated in Squalane 0707.

FIGS. 4a-b—detail the degradation of Avobenzone in presence of single carotenoids

FIG. 4a—Percent of initial avobenzone remaining in the presence of various carotenoids compositions, after 18 hours of combined UVA+B exposure, illuminants combined at 4:2 ratio.

FIG. 4b—Percent of initial avobenzone remaining in the presence of various carotenoids compositions, after 10 hours of combined UVA+B+C exposure, illuminants combined at 3:2:1 ratio. Phytoene was tested at 1.3 mg/mL; Phytofluene was tested at 0.3 mg/mL; zeta-carotene and lycopene were tested at 1.6 mg/mL, all in squalane medium.

FIG. 5—Percent of initial avobenzone remaining in the presence of compositions of varying phytoene/phytofluene ratios, after 10 hours of combined UVA+B+C exposure, illuminants combined at 3:2:1 ratio. Levels tested: a ‘low’ level: 0.3 mg/mL, ‘high’ level 1.3 mg/mL. Phytoene, phytofluene or mixtures thereof were each tested at both ‘high’ and ‘low’ levels, and in combinations (high PE+low PF, low PE+high PF).

FIGS. 6a-g—Degradation of sunscreen actives other than avobenzone in the presence of phytoene and phytofluene. Percentage of initial sunscreen active remaining in the presence of compositions of increasing phytoene/phytofluene concentrations. Phytoene and phytofluene mixture is represented by the exemplary stabilizing composition IBR-TCLC® Concentrated in Squalane 0707. Sunscreen Actives: Avobenzone, 2-ethylhexyl-4-metyhoxycinnamate (EMC) and dibenzoylmethane (DBM)

FIG. 6a—Avobenzone; 18 hours of UVA exposure

FIG. 6b—DBM; 18 hours of UVA exposure

FIG. 6c—DBM; 18 hours of UVB exposure

FIG. 6d—EMC; 18 hours of UVB exposure

FIG. 6e—Avobenzone; 5 hours of UVC exposure

FIG. 6f—DBM; 5 hours of UVC exposure

FIG. 6g—EMC; 5 hours of UVC exposure

FIG. 7—Stabilization of phenol under UV irradiation in presence of P&P. Percent of initial phenol remaining in the presence of compositions of 0.25% TiO2 and increasing phytoene/phytofluene concentrations; UVA exposure.

FIG. 8: Degradation of avobenzone in polydecene oil under sunlight: percent of initial avobenzone remaining in the presence of increasing phytoene/phytofluene concentrations; sunlight exposure.

FIG. 9 Comparison of SPF and UVA-PF of creams containing avobenzone (703-1) and avobenzone with phytofluene (703-2) before and after UVA irradiation of 10 and 20 J/cm2 using Suntest®

FIG. 10. Absorbance spectra of phytoene (λmax 286 nm) and phytofluene (λmax 348 nm).

### Definitions

“Sunscreen composition”, is a cream, lotion, spray, gel, lipstick or other topical composition, comprising at least one sunscreen active (also known as sunscreen), used for UV protection of the skin.

“UV protection” is a definition for protection capability against the full UV spectra including UVA, UVB and UVC.

“Sunscreen active” or “sunscreen active ingredient” or simply “sunscreen” is a molecule added to a sunscreen composition in order to achieve screening of UV light and thus protect the skin. As sunscreens are intended for the absorption of UV light, they are also called “UV actives” or UV-filters”.

### DETAILED DESCRIPTION OF THE INVENTION

The present invention provides innovative methods of stabilization of sunscreens and sunscreen compositions comprising at least one sunscreen active, exhibiting enhanced stability of the sunscreen actives and sunscreen compositions towards sunlight, free radical attack or combinations thereof The enhanced stability is preserved also in the presence of inorganic sunscreens like ZnO and TiO2.

The enhanced stability of the sunscreens and sunscreen compositions is achieved by addition of a mixture of phytoene, phytofluene and optionally of at least one additional natural or synthetic carotenoid.

Phytoene and phytofluene, optionally with the addition of at least one additional carotenoid, stabilize sunscreen compositions and sunscreen actives by one or more of following mechanisms:

1. Stabilization of sunscreen actives (such as avobenzone) from degradation by sunlight due to UV absorption
2. Quenching of free radicals thus preventing the oxidative damage generated by inorganic sunscreen actives when irradiated with UV light.
3. Boosting the SPF and UVA-PF of sunscreen actives (such as avobenzone

### Results and Discussion

The present invention provides methods of sunscreen actives in general and avobenzone in particular, by addition to the sunscreen active or the sunscreen composition of mixtures of phytoene/phytofluene, and optionally other carotenoids.

The stabilizing effect of carotenoids in general, and that of the colorless carotenoids phytoene/phytofluene (P&P) in particular on sunscreens was investigated by photo-stability testing, as described in Example 1.

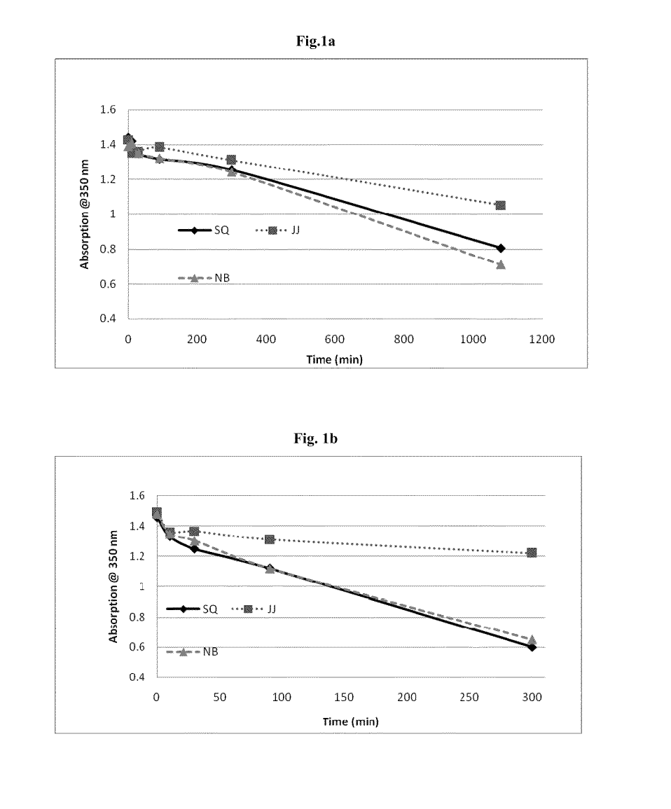

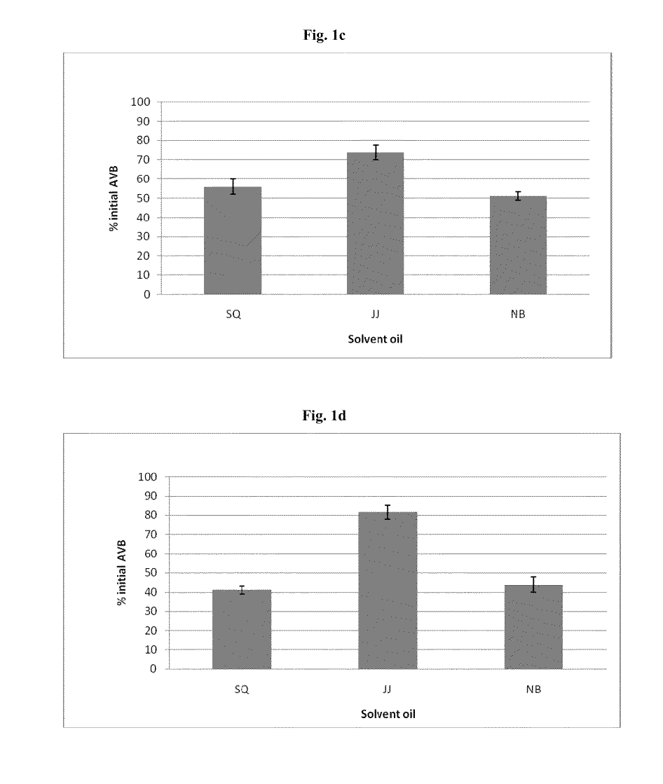

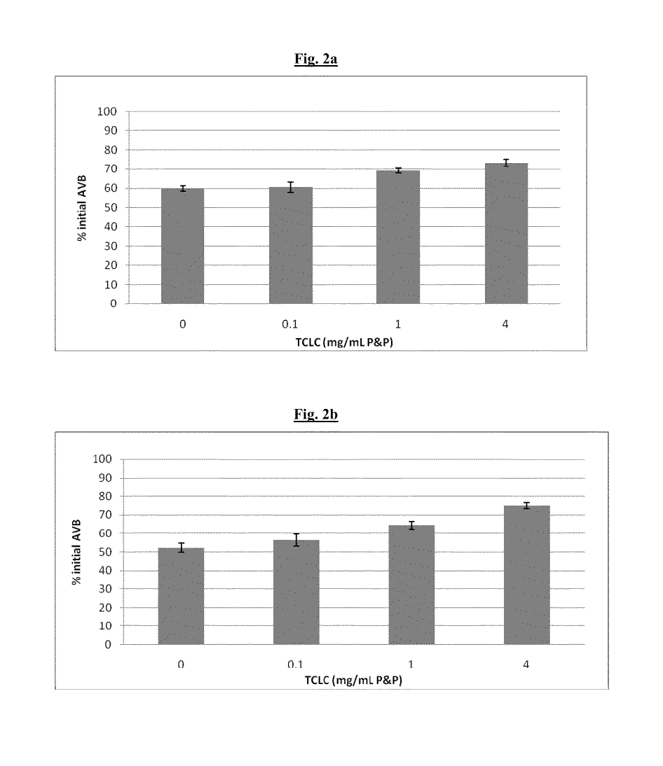

We have shown (see Example 2) that non-stabilized avobenzone undergoes degradation under UVA and UVC irradiation in Hydrogenated polydecene, Squalane, and Jojoba oil (colorless). (See also FIG. 1a-d).

The impact of various solvents on the avobenzone's degradation under UV irradiation the above three solvents was investigated (see Example 2). It was surprisingly found that the avobenzone degradation in jojoba oil is significantly slower as compared to the other media (see FIGS. 1a-d). It is unlikely that this effect is due to direct protection of avobenzone by Jojoba oil (no significant absorption in the relevant UV range). Avobenzone's degradation is known to proceed more slowly and in different ways in polar solvents than in non-polar ones.

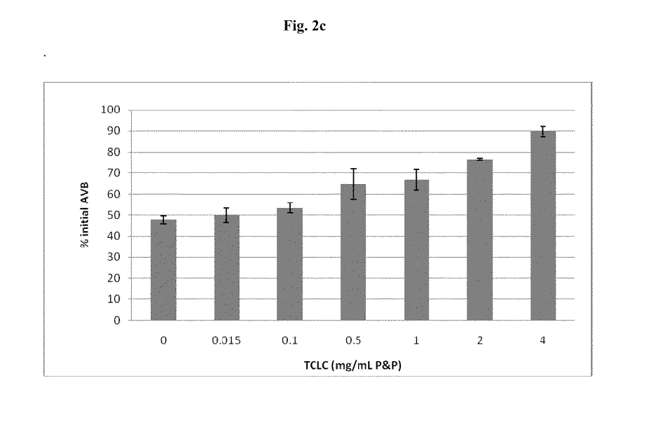

We have also shown (Example 3) that irradiation of avobenzone under different single UV domains degrades avobenzone differently (see FIG. 2a-c). Phytoene/phytofluene mixtures are able to stabilize avobenzone under different irradiation conditions.

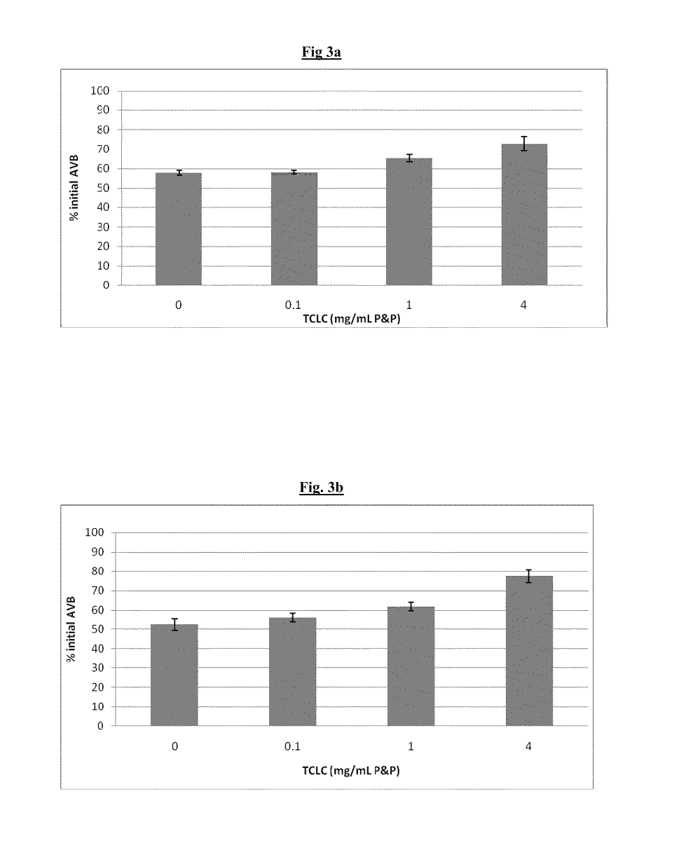

When avobenzone was irradiated under combined UV domains (Example 4, FIG. 3a-b), mimicking real-use situations under sunlight (sunlight being a broad-spectrum illuminant including all three UV domains), we have found that data correlates well with data obtained with single wavelength domains, showing significant protection of avobenzone by phytoene, phytofluene or mixtures thereof at high levels under combined UVA+UVB+UVC irradiation, and somewhat more moderate protection under UVA+UVB irradiation.

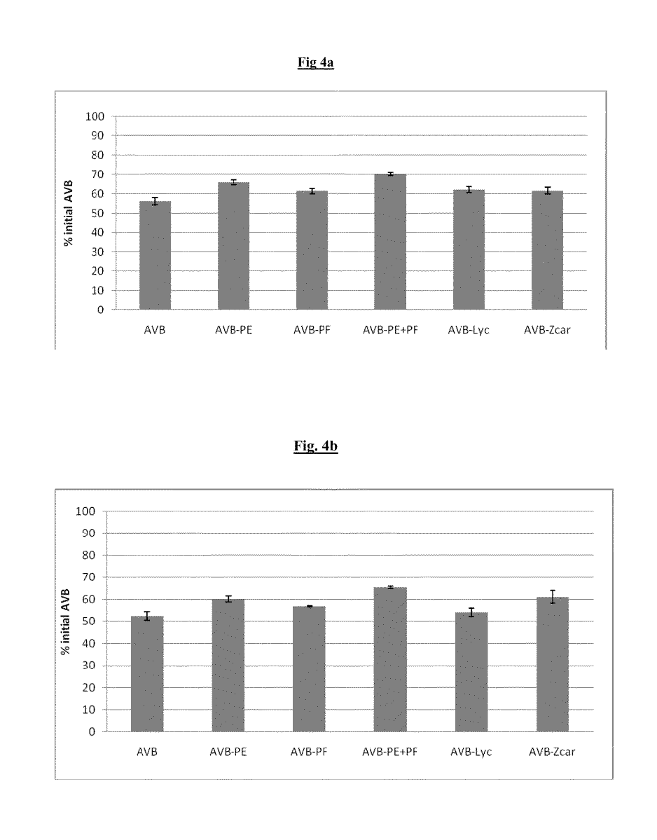

The stabilizing effect of single carotenoids on avobenzone was investigated (Example 5). Irradiation was carried out with combined UVA+UVB (4:2, 18 h) and UVA+UVB+UVC (3:2:1, 10 h). We have surprisingly found that phytoene and phytofluene had a very good stabilizing effect. Additive effects were observed for the combination of phytoene and phytofluene. We have found that PE and PF appear to have roughly comparable activities under our experimental conditions. Lycopene showed a slight protective activity under UVA+UVB, but no significant activity once UVC was introduced. Zeta-carotene also showed some protective activity. See FIG. 4a-b.

The impact of the ratio of phytoene to phytofluene in phytoene and phytofluene mixtures on the avobenzone stabilization was investigated (see Example 6 and FIG. 5). Additive effects were observed for the combination of phytoene and phytofluene. Protective activity of phytoene and phytofluene appears to be level-dependent. Phytoene and phytofluene appear to have roughly comparable activities under these experimental conditions.

Sunscreens other than avobenzone are also stabilized by phytoene and phytofluene mixtures. Thus, 2-ethylhexyl-4-metyhoxycinnamate (EMC) and dibenzoylmethane (DBM) were irradiated in presence of a commercial P&P mixture, IBR-TCLC® Concentrated in Squalane 0707.

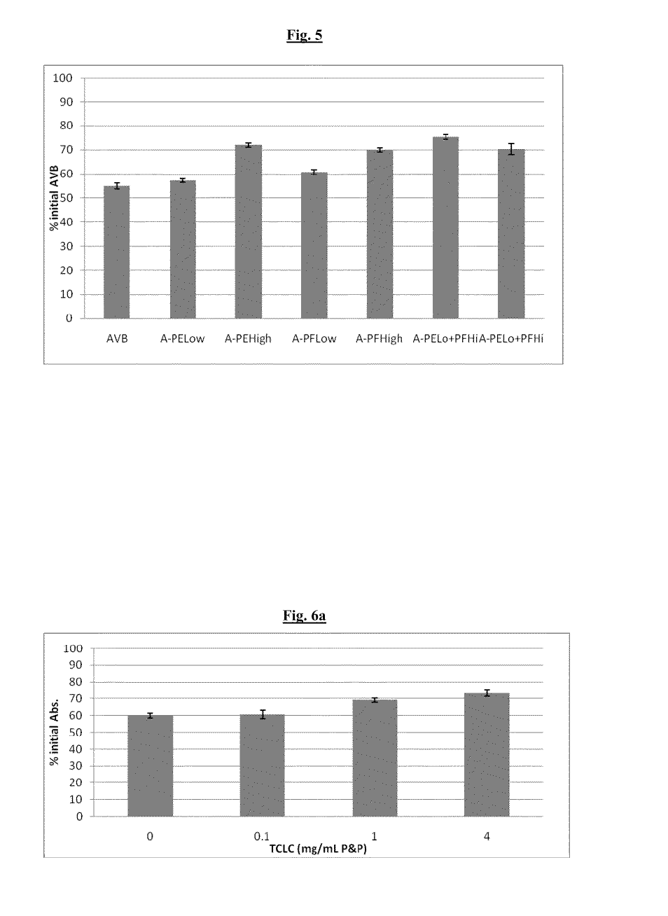

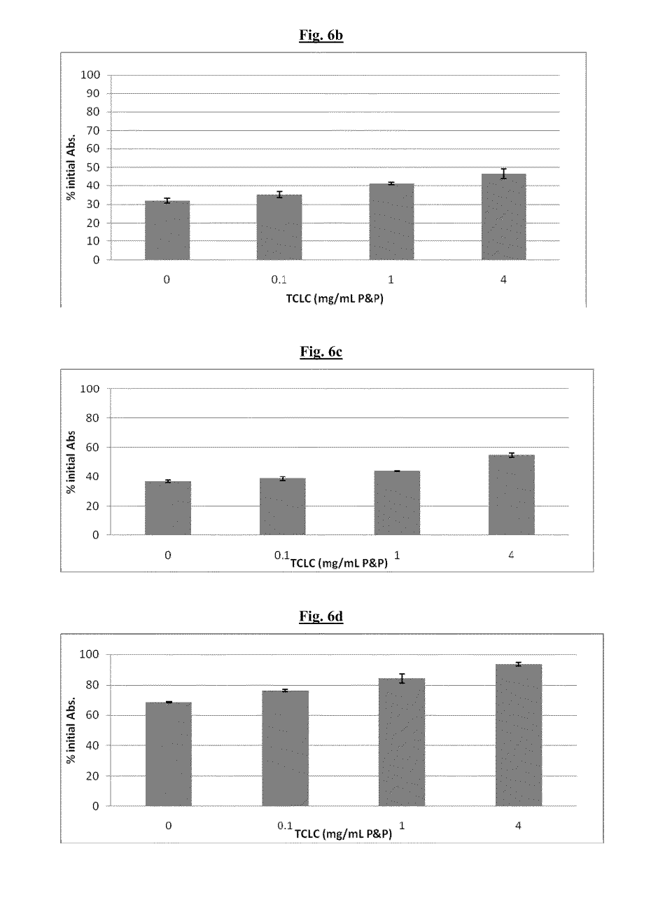

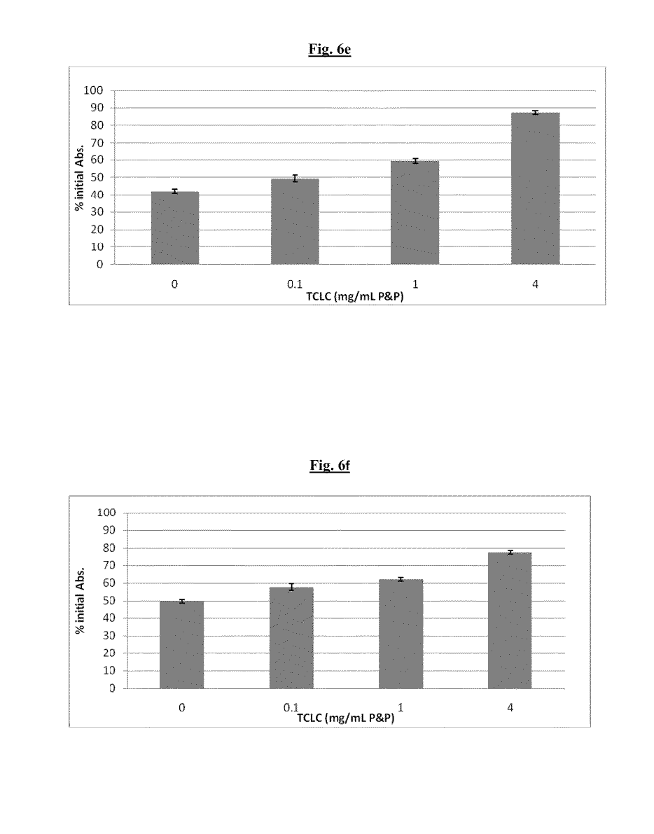

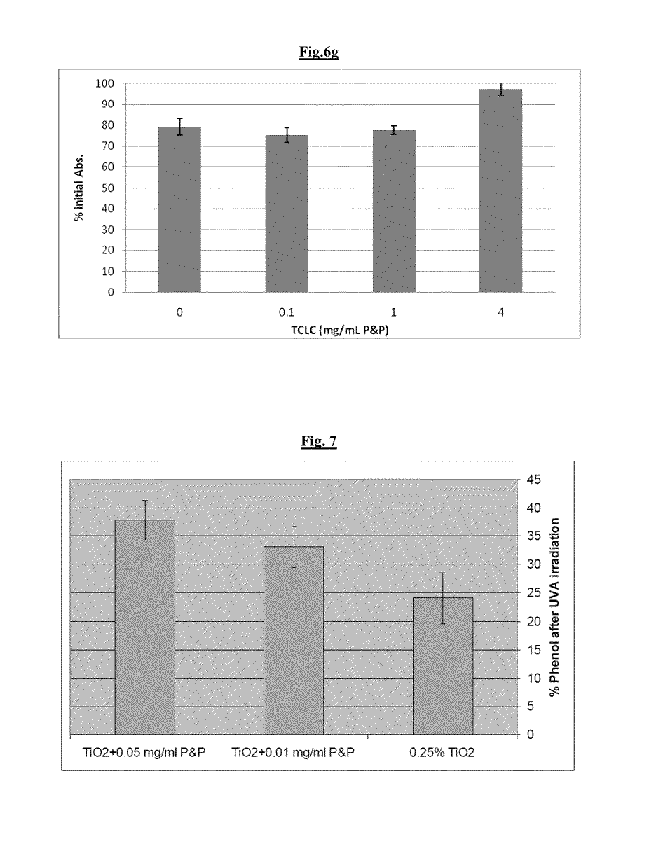

Irradiation took place as described above in the case of avobenzone (18 h UVA; 18 h UVB; 5 h UVC respectively) (see FIG. 6a-f and Example 7). We have found that phytoene and phytofluene have a stabilizing effect on these other sunscreens as well.

Phenol ,a highly sensitive compound to free radicals decomposition, was UV irradiated in the presence of TiO2 (0.25%), with increased levels of additional phytoene and phytofluene: TiO2 0.25%+0.01 mg/ml phytoene and phytofluene vs. TiO2 0.25%+0.05 mg/ml phytoene and phytofluene. Increased levels of phytoene and phytofluene increased the stability and resistance of phenol to degradation by UV irradiated TiO2 generated free radicals. See FIG. 7.

We have found that when a 2% solution of avobenzone in oil (polydecene) was prepared and exposed 10 minutes to sunlight, avobenzone levels were reduced by 36%. In the presence of 0.015% phytoene and Phytoene and phytofluene mixture is represented by the exemplary stabilizing composition IBR-TCLC® 0701.

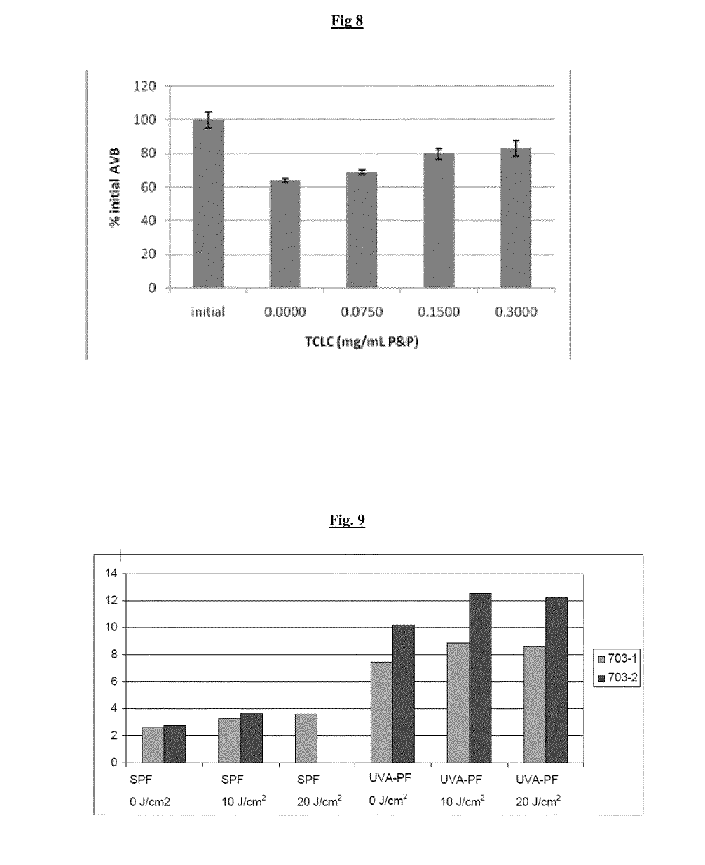

The photo decomposition of avobenzone was only 17%, retaining 50% more avobenzone than without stabilizer.These results show that phytoene and phytofluene were able to both stabilize avobenzone (FIG. 8) and quench the free radicals generated from irradiation of TiO₂ (see FIG. 2 and Example 9) thereby increasing the sunscreens safety, stability and ultimate efficacy.

A basic cream containing 3% avobenzone to which phytoene and phytofluene (0.0075%), was added was tested for stability. The phytoene and phytofluene stabilizing effect was confirmed (See Example 10).

While natural or synthetic phytoene, phytofluene or mixtures thereof effectively stabilize sunscreens, adding at least one more carotenoid to the sunscreen to the composition is conducive to an even better stabilization. The additional carotenoid is selected from the group consisting of zeta-carotene, beta-carotene, lycopene, zeaxanthin, astaxanthin, cataxanthin, zeaxanthin, lutein, canthaxanthin and the like, and any combination thereof The ratio between the phytoene, phytofluene or mixtures thereof to the other carotenoid is from about 1:1 to about 50:1, alternatively from 1:1 to about 1:50. The composition optionally comprises inactive ingredients selected from the group consisting of, but not limited to anti-oxidants, anti-inflammatory agents and vitamins.

According to the instant invention, the stabilization of sunscreens and sunscreen compositions is achieved by the addition of a stabilizing composition to the sunscreen or sunscreen composition. Several non-limiting examples of such stabilizing compositions are detailed below:

1. A squalane based composition comprising 1 part of a phytoene, phytofluene or mixtures thereof, wherein phytoene, phytofluene or mixtures thereof are present in 4.3/1 ratio, and 50 parts of lycopene.
2. A squalane based composition comprising 1 part of a phytoene, phytofluene or mixtures thereof, wherein phytoene, phytofluene or mixtures thereof are present in 4.3/1 ratio, and 50 parts of β carotene.
3. A squalane based composition comprising 1 part of a phytoene, phytofluene or mixtures thereof, wherein phytoene, phytofluene or mixtures thereof are present in 4.3/1 ratio, and 50 parts of a β carotene and lycopene mixture.
4. A squalane based composition comprising 1 part of a phytoene, phytofluene or mixtures thereof, wherein phytoene, phytofluene or mixtures thereof are present in 4.3/1 ratio, and 50 parts of a β carotene and zeta carotene mixture
5. A squalane based composition comprising 1 part of a phytoene, phytofluene or mixtures thereof, wherein phytoene, phytofluene or mixtures thereof are present in 4.3/1 ratio, and 50 parts of a lycopene, β carotene and zeta carotene
6. A squalane based composition comprising 50 parts of a phytoene, phytofluene or mixtures thereof, wherein phytoene, phytofluene or mixtures thereof are present in 4.3/1 ratio, and 1 part of lycopene.

7. A squalane based composition comprising 50 parts of a phytoene, phytofluene or mixtures thereof, wherein phytoene, phytofluene or mixtures thereof are present in 4.3/1 ratio, and 1 part of β carotene.

8. A squalane based composition comprising 50 parts of a phytoene, phytofluene or mixtures thereof, wherein phytoene, phytofluene or mixtures thereof are present in 4.3/1 ratio, and 1 part of a β carotene and lycopene mixture

9. A squalane based composition comprising 50 parts of a phytoene, phytofluene or mixtures thereof, wherein phytoene, phytofluene or mixtures thereof are present in 4.3/1 ratio, and 1 part of a β carotene and zeta carotene mixture

10. A squalane based composition comprising 50 parts of a phytoene, phytofluene or mixtures thereof, wherein phytoene, phytofluene or mixtures thereof are present in 4.3/1 ratio, and 1 part of a lycopene, β carotene and zeta carotene.

The mixtures of additional carotenoids (lycopene, β-carotene, zeta carotene), to be added to the P&P mixtures are present in ratios between 1:10 and 10:1.

In a preferred embodiment, the instant invention provides sunscreen compositions comprising at least one sunscreen active, wherein said sunscreen active is stabilized by the addition to the composition of a mixture of phytoene and phytofluene.

The ratio between phytoene, phytofluene or mixtures thereof is between 200:1 to 1:200 w/w %, preferably between 50:1 to 1:50 w/w % or more preferably between 10:1 to 1:10 w/w %.

In another embodiment, the ratio between phytoene, phytofluene or mixtures thereof is around 10:1 w/w % or around 1:1 w/w %.

Phytoene, phytofluene or mixtures thereof may be in almost pure form, so that the ratio between phytoene, phytofluene or mixtures thereof is between 200:1 to 100:1 w/w % or higher or between 1:200 to 1:100 w/w % or lower.

In another embodiment, single natural or synthetic colorless carotenoids phytoene or phytofluene (instead of their mixtures) are used for the stabilization of sunscreens or sunscreen compositions.

In a preferred embodiment, there are provided sunscreens and sunscreen compositions stabilized with natural or synthetic single colorless carotenoids phytoene or phytofluene.

In another preferred embodiment, there are provided methods for stabilization of sunscreens and sunscreen compositions by addition of natural or synthetic single colorless carotenoids phytoene or phytofluene.

Phytoene/phytofluene mixtures are commercially available from IBR, Israeli Biotechnology Research Ltd. An IBR commercial product line sold under the trade name IBR-Phyto(flu)ene®. One of the products in this line is IBR-TCLC® (Tomato Colorless Carotenoids), which is a clear yellow to yellowish liquid containing 0.5 to 1.0 mg/ml phytoene and phytofluene. Other IBR phytoene and phytofluene products, IBR-CLC® and IBR-AAC®, which are sourced from algae, are available as oil solutions in various solvents and concentrations: IBR-CLC® 0401, IBR-CLC® concentrated 0402, IBR-CLC® concentrated in Squalane 0404, IBR-CLC® in Jojoba Oil 0407, IBR-AAC® 0501, IBR-TCLC® 0701, IBR-TCLC® in Squalane 0702, IBR-TCLC® in Jojoba Oil 0705.

In a preferred embodiment, there are provided sunscreen compositions stabilized by addition of phytoene/phytofluene containing products selected from but not limited to IBR-Phyto(flu)ene® product line. IBR-TCLC® (Tomato Colorless Carotenoids), IBR-CLC®, IBR-AAC®, IBR-CLC® 0401, IBR-CLC® concentrated 0402, IBR-CLC® concentrated in Squalane 0404, IBR-CLC® in Jojoba Oil 0407, IBR-AAC® 0501, IBR-TCLC® 0701, IBR-TCLC® in Squalane 0702 and IBR-TCLC® in Jojoba Oil 0705 or combinations thereof and optionally at least one natural or synthetic carotenoid other than phytoene/phytofluene.

In another preferred embodiment, there are provided methods for the stabilization of sunscreens or sunscreen compositions by addition of phytoene/phytofluene containing products selected from but not limited to IBR-Phyto(flu)ene® product line. IBR-TCLC® (Tomato Colorless Carotenoids), IBR-CLC®, IBR-AAC®, IBR-CLC® 0401, IBR-CLC® concentrated 0402, IBR-CLC® concentrated in Squalane 0404, IBR-CLC® in Jojoba Oil 0407, IBR-AAC® 0501, IBR-TCLC® 0701, IBR-TCLC® in Squalane 0702 and IBR-TCLC® in Jojoba Oil 0705 or combinations thereof and optionally at least one natural or synthetic carotenoid other than phytoene/phytofluene.

It was surprisingly found that the solvents have an important impact on the sunscreens' stabilization with carotenoids, and especially with phytoene/phytofluene mixtures.

In one embodiment, there is provided a method for stabilization of sunscreens or sunscreen compositions, by addition of IBR-TCLC® in Jojoba Oil 705.

In another embodiment, sunscreen compositions are stabilized by addition of 0.1% to 2% of IBR-TCLC® solution.

In one embodiment, at least one of the sunscreen actives in the compositions of this invention is avobenzone. In another embodiment, at least one of the sunscreen actives is avobenzone and the other optional sunscreen actives are selected from the group comprising Oxybenzone, Sulisobenzone, Dioxybenzone, Octabenzone, p-aminobenzoic acid, Pamidate O, phenylbenzimidazole sulfonic acid, cinoxate, dioxybenzone, oxybenzone, homosalate, menthyl anthranilate, octocrylene, octyl methoxy cinnamate, octyl salicylate, sulisobenzone, trolamine salicylate, Ecamsule, titanium oxide, zinc oxide, 4-methylbenzylidene camphor, Tinosorb M, Tinosorb S, Neo Heliopan AP, Memoryl XL, benzophenone-9, benzophenone-3, benzophenone-12, benzophenone-4, benzophenone-8, Uvinul T 150, Uvinul A Plus, Uvasorb HEB, Parsol SLX, isopentenyl-4-methoxycinnamate or combinations thereof.

The stabilized compositions of the instant invention may further comprise at least one additional natural or synthetic carotenoid such as, but not limited to zeta-carotene, beta-carotene, lycopene, zeaxanthin, astaxanthin, cataxanthin, zeaxanthin, lutein and canthaxanthin mixtures thereof.

The ratio between the phytoene, phytofluene or mixtures thereof to the other carotenoid ranges from about 1:1 to about 50:1 or from 1:1 to about 1:50. Alternatively, the ratio between the phytoene, phytofluene or mixtures thereof to the other carotenoid is from about 1:1 to about 10:1 or from 1:1 to about 1:10.

In a preferred embodiment, the stabilization of the sunscreen active in the composition prevents degradation of the sunscreen active as a result of sunlight, free radical attack or combinations thereof

In another embodiment, the stabilization of the sunscreen prevents degradation of the sunscreen active in the presence of inorganic sunscreens selected from the group comprising ZnO, TiO2 as a result of sunlight, free radical attack or combinations thereof.

The prevention of degradation and enhancement of stability may be achieved in a hydrophobic or oil solution.

Said prevention of the degradation and enhancement of stability may be achieved in a cosmetic composition.

The compositions of this invention may further comprise at least one antioxidant, leading to enhanced stability to sunlight.

The sunscreen compositions may further comprise additional pharmaceutically or cosmetically acceptable ingredients selected from the group comprising antioxidants, anti-inflammatory agents and vitamins.

In a preferred embodiment, the addition to the composition of a mixture of phytoene, phytofluene and optionally at least one additional natural or synthetic carotenoid is conducive to an improved SPF and/or UVA-PF of the composition.

The compositions of the instant invention may be in the form of a cream, lotion, spray, gel, roll-on, lipstick or other pharmaceutically or cosmetically acceptable topical product.

Said compositions may be packed in a suitable packing made of materials enabling its use and preserving its stability.

In one embodiment there is provided a method for enhancing the stability and preventing the degradation of sunscreen actives by sunlight or UV light by addition to said sunscreen actives of a mixture of phytoene and phytofluene.

In another embodiment, the sunscreen active stabilization is achieved by the protection of the active and the sunscreen composition from the deleterious effects of UV light, including UVA, UVB, UVC or combinations thereof, as such or as part of the natural daylight.

In a preferred embodiment, the stabilization of the sunscreen actives and sunscreen compositions is conducive to the improvement of their effectiveness and reduction of their toxicity.

The sunscreen stabilization fulfils, in addition to a formulative need, also an important industry safety need.

Said method may use a mixture between phytoene and phytofluene in a ratio between 200:1 to 1:200 w/w % or between 50:1 to 1:50 w/w %, preferably between 10:1 to 1:10 w/w %.

Alternatively, said method uses a ratio between phytoene and phytofluene around 10:1 w/w % or around 1:1 w/w %.

The method of stabilization may use almost pure compounds, so that the ratio between phytoene and phytofluene is between 200:1 to 100:1 w/w % or higher or between 1:200 to 1:100 w/w % or lower.

Alternatively, the said method of stabilization may use single natural or synthetic phytoene or phytofluene.

The method of stabilization of the sunscreen active is effective on compositions wherein at least one of the sunscreen actives is avobenzone, or when at least one of the sunscreen actives is avobenzone and the other sunscreen actives are selected from the group comprising p-aminobenzoic acid, Pamidate O, phenylbenzimidazole sulfonic acid, cinoxate, dioxybenzone, oxybenzone, homosalate, menthyl anthranilate, octocrylene, octyl methoxy cinnamate, octyl salicylate, sulisobenzone, trolamine salicylate, Ecamsule, titanium oxide, zinc oxide, 4-methylbenzylidene camphor, Tinosorb M, Tinosorb S, Neo Heliopan AP, Memoryl XL, benzophenone-9, Uvinul T 150, Uvinul A Plus, Uvasorb HEB, Parsol SLX, isopentenyl-4-methoxycinnamate or combinations thereof .

Alternatively, the method of stabilization includes further addition of a natural or synthetic carotenoid selected from the group comprising zeta-carotene, beta-carotene, lycopene, zeaxanthin, astaxanthin, cataxanthin, zeaxanthin, lutein, canthaxanthin or mixtures thereof.

The method of stabilization of the sunscreen active prevents degradation of the sunscreen active as a result of sunlight, free radical attack or combinations thereof.

Said method of stabilization of the sunscreen actives prevents degradation of the sunscreen active in the presence of inorganic sunscreens selected from the group comprising ZnO, TiO2 as a result of sunlight, free radical attack or combinations thereof This may be achieved in a hydrophobic or oil solution. Same may be achieved in a cosmetic composition.

The stabilization of the sunscreen actives in the sunscreen compositions is conducive to an enhanced physical and chemical stability of the sunscreen composition itself (see Example 10 and Example 12 detailing a typical sunscreen composition).

In a preferred embodiment, there is provided a method of protection of a human in need thereof from UV light, by application to its skin of an effective amount of a composition comprising phytoene, phytofluene and optionally at least one additional carotenoid.

In another embodiment, there is provided a kit comprising at least one packed composition of any of the compositions of the instant invention and instructions for use.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

### REFERENCES

1. L. von Oppen-Bezalel. Lightening, Boosting and Protecting with Colorless Carotenoids. Cosmetics & Toiletries® magazine. March 2009. Vol. 124, No. 3: 66-75.
2. L. von Oppen-Bezalel. Round-up: Colourless carotenoids may protect skin against inflammation and UV radiation. H&PC Today. n2/2008 p: 31-34.
3. L. von Oppen-Bezalel. UVA, A Main Concern in Sun Damage: Protection from the Inside and Outside with Phytoene, Phytofluene, the Colorless Carotenoids and more. SÖFW-Journal, 11-2007
4. Liki von Oppen-Bezalel, Aviv Shaish. Application of the Colorless Carotenoids, Phytoene, and Phytofluene in Cosmetics, Wellness, Nutrition, and Therapeutics. In The alga Dunaliella: Biodiversity, Physiology, Genomics & Biotechnology Eds. Ami Ben-Amotz, Juergen Polle, and Subba Rao, released May 2009 by Science Publishers, Enfield, NH, USA (2009)
5. Liki von Oppen-Bezalel, Eyal Lerner, Dale G. Kern, Bryan Fuller, Etienne Soudant, Aviv Shaish. IBR-CLC®, Colorless Carotenoids: Phytoene and Phytofluene from Unicellular Algae—Applications in Cosmetics, Wellness and Nutrition. Fragrance Journal, 2006, VOL 34; PART 3, pages 48-53.
6. Vielhaber G, Grether-Beck S, Koch O, Johncock W, Krutmann J (March 2006). “Sunscreens with an absorption maximum of > or =360 nm provide optimal protection against UVA1-induced expression of matrix metalloproteinase-1, interleukin-1, and interleukin-6 in human dermal fibroblasts”. Photochem Photobiol Sci 5(3): 275-82.
7.Chatelain E, Gabard B. (September 2001). “Photostabilization of Butyl methoxydibenzoylmethane (Avobenzone) and Ethylhexyl methoxycinnamate by Bis-ethylhexyloxyphenol methoxyphenyl triazine (Tinosorb S), a new UV broadband filter”. Photochem Photobiol 74(3): 401-6.
8.Tarras-Wahlberg N, Stenhagen G, Larko O, Rosen A, Wennberg A M, Wennerstrom O (October 1999). “Changes in ultraviolet absorption of sunscreens after ultraviolet irradiation”. J Invest Dermatol 113(4): 547-53
9.Wetz F, Routaboul C, Denis A, Rico-Lattes I (Mar-Apr 2005). “A new long-chain UV absorber derived from 4-tert-butyl-4′-methoxydibenzoylmethane: absorbance stability under solar irradiation”. J Cosmet Sci 56(2): 135-48.
10. CTFA letter re: Tentative Final Monograph for OTC Sunscreen
11. Bonda C., Steinberg D. C. (2000). “A new photostabilizer for full spectrum sunscreens”. Cosmet. Toiletries 115(6): 37-45.
12. http://www.dsm.com/en_US/downloads/dnp/Parsol_SLX_Skin.pdf
13. Chaudhuri R K, Lascu Z, Puccetti G, Deshpande A A, Paknikar S K (May-June 2006). “Design of a photostabilizer having built-in antioxidant functionality and its utility in obtaining broad-spectrum sunscreen compositions.”. Photochem Photobiol 82(3): 823-8.
14. http://www.hallstar.com/techdocs/Polycrylene&CorapanTQAvobenzoneStabilization.pdf
15. Scalia S, Simeoni S, Barbieri A, Sostero S (November 2002). “Influence of hydroxypropyl-beta-cyclodextrin on photo-induced free radical production by the sunscreen agent, butyl-methoxydibenzoylmethane.”. J Pharm Pharmacol 54(11): 1553-8.

### 16. Warwick L. Morison, M. D. (Mar. 11, 2004.). “Photosensitivity”. The New England Journal of Medicine 350: 1111-1117.

17. “Sunscreen Drug Products for Over-the-Counter use; Marketing Status of Products Containing Avobenzone; Enforcement Policy” (PDF). US Food and Drug Administration. 1997-04-30. 23354. http://www.fda.gov/cder/otcmonographs/Sunscreen/sunscreen_avobenzone_enforcpolicy_{—}19970420.pdf. Retrieved on Jun. 3, 2007.
18. Stability Study of Avobenzone with Inorganic Sunscreens, Kobo Products Poster, 2001, Online version
19. Wakefield G, Lipscomb S, Holland E, Knowland J (July 2004). “The effects of manganese doping on UVA absorption and free radical generation of micronized titanium dioxide and its consequences for the photo-stability of UVA absorbing organic sunscreen components.” Photochem. Photobiol. Sci. 3(7): 648-52.

### EXAMPLES

The following examples further illustrate the invention but, of course, should not be construed as in any way limiting its scope.

### Example 1

### Photo-Stability Testing

Key elements for this test are: consistent irradiation (known and consistent light source and positioning of samples with respect to the light source); optimal exposure of the sample to UV light (thin but consistent layer sample, in a flat vessel with no high walls which could screen out UV light); consistent sampling (especially recovery of irradiated material for analysis). Under the instant method, irradiation takes place in the Innova 4340 incubator, fitted with UV tubes (UVC: Osram Puritec-HNS3OVG13-G30T8/OF; UVB: Voltarc E312/F30T8; UVA: Sylvania F30T8/350BL)—allowing for intense, known, reproducible and even irradiation (a known illuminant is the single source of light—a wide light source and the incubator's metallic walls also help achieve even illumination). Samples are exposed in Brand plastic vial caps made of polyethylene, 26 mm diameter, 5 mm wall height, 400 μL sample volume, in triplicates, generally introduced as solutions in suitable oil carriers)—enabling irradiation of a relatively even and thin layer while allowing consistent sample recovery.

Analysis of the avobenzone content of the samples was done by UV spectroscopy (absorption @ 350 nm for avobenzone; sample dilution ×400 in hexanes). As phytofluene absorbs in the UV range relevant to our measure of avobenzone, blank TCLC samples (i.e. without avobenzone) were irradiated in parallel with test samples and absorption @ 350 nm was measured before and after irradiation. This absorption was deducted from the avobenzone absorption data, producing the corrected data shown here (unless noted otherwise).

### Example 2

### Degradation of Avobenzone in Various Solvents

Degradation of Avobenzone was measured vs. time under UVA and UVC irradiation in Hydrogenated polydecene, Squalane, and Jojoba oil (colorless). (See FIGs. 1a-d).

Degradation in jojoba oil is significantly slower as compared to the other media. It is unlikely that this effect is due to direct protection of avobenzone by Jojoba oil (no significant absorption in the relevant UV range).

### Example 3

### Irradiation Under Single UV Domains

Samples were irradiated overnight (14 h) with UVA; overnight (18 h) with UVB; or for 5 h with UVC. See FIGS. 2a-c.

The results of these irradiation tests show that different UV wavelengths degrade avobenzone differently. Phytoene/phytofluene mixtures are able to stabilize avobenzone under different irradiation conditions.

### Example 4

### Irradiation Under Combined UV Domains

To confirm the trends observed with single UV domains as well as to further approximate the real-use situation (sunlight being a broad-spectrum illuminant including all three UV domains), avobenzone was irradiated in the presence of TCLC® in Squalane 0702 with combined UVA+UVB (illuminants combined 4:2, 18 h irradiation,) or UVA+UVB+UVC (illuminants combined 3:2:1, 10 h irradiation). See FIGS. 3a-b.

The above data correlates with data obtained with single wavelength domains, showing significant protection of avobenzone by P&P at high levels under combined UVA+UVB+UVC irradiation, and somewhat more moderate protection under UVA+UVB irradiation

### Example 5

### Stabilization of Avobenzone in Presence of Single Carotenoids

Based on the above results, degradation of avobenzone was next measured in the presence of single carotenoids, specifically: phytoene (PE), phytofluene (PF), zeta-carotene (ZC) and Lycopene (LY).

Carotenoids levels: for the purposes of these experiments, phytoene was tested at 1.3 mg/mL; phytofluene was tested at 0.3 mg/mL; zeta-carotene and lycopene were tested at 1.6 mg/mL.

Irradiation was carried out with combined UVA+UVB (4:2, 18 h) and UVA+UVB+UVC (3:2:1, 10 h). See FIG. 4a-b.

Additive effects were observed for the combination of phytoene and phytofluene.

Lycopene showed a slight protective activity under UVA+UVB, but no significant activity once UVC was introduced. Zeta-carotene also showed some protective activity (roughly equivalent to phytoene.

### Example 6

### Effect of Phytoene/Phytofluene Levels and Ratio Variations on Stabilization

In order to complete the picture above, different levels and ratios of phytoene, phytofluene or mixtures thereof were tested for their protective activity vs. degradation of avobenzone, under combined UVA+UVB+UVC irradiation (10 h).

Levels tested: a “low” level was defined as 0.3 mg/mL, along with a “high” level of 1.3 mg/mL. Phytoene, phytofluene or mixtures thereof were each tested at both “high” and “low” levels, and in combinations (high PE+low PF, low PE+high PF). (The results are summarized in the FIG. 5)

Additive effects were observed for the combination of phytoene and phytofluene.

Protective activity of PE and PF appears to be level-dependent.

PE and PF appear to have roughly comparable activities under these experimental conditions.

### Example 7

### Stabilization of Sunscreen Ingredients Other Than Avobenzone in the Presence of TCLC

In order to obtain a broader picture of protective effects of phytoene, phytofluene or mixtures thereof versus UV-induced degradation of the various sunscreen ingredients, the following ingredients were irradiated in the presence of phytoene, phytofluene or mixtures thereof (as TCLC), along with avobenzone as reference leg: 2-ethylhexyl-4-metyhoxycinnamate (EMC) and dibenzoylmethane (DBM), representing a simple model compound for ingredients with similar structures to avobenzone. Irradiation took place as described above in the case of avobenzone (18 h UVA; 18 h UVB; 5 h UVC respectively). See FIGS. 6a-f.

### Example 8

### Stabilization of Phenol Under UV Irradiation in Presence of P&P

Phenol (a highly sensitive compound to free radicals decomposition) was UV irradiated in the presence of TiO2 (0.25%), with increased levels of additional phytoene and phytofluene: TiO2 0.25%+0.01 mg/ml phytoene and phytofluene vs. TiO2 0.25%+0.05 mg/ml phytoene and phytofluene. Increased levels of phytoene and phytofluene increased the stability and resistance of phenol to degradation by UV irradiated TiO2. See FIG. 7.

### Example 9

### Degradation of Avobenzone Under Sunlight

A 2% solution of avobenzone in oil (polydecene) was prepared and exposed 10 minutes to sunlight (Israel, June, 4 pm) The same concentration of avobenzone was mixed with increasing levels of phytoene and phytofluene (0%, 0.0037%, 0.0075% and 0.015%) in the polydecene solutions and exposed 10 minutes to the sun. Levels of avobenzone were quantified using UV-Visible spectrophotometer and calculated based on the extinction coefficient value 1618 of avobenzone in hexane at λ-350 nm.

Following 10 minutes exposure to sunlight, avobenzone levels were reduced by 36%. In the presence of 0.015% phytoene and phytofluene the photo decomposition of avobenzone was only 17%, retaining 50% more avobenzone than without stabilizer.These results show that phytoene and phytofluene were able to both stabilize avobenzone (FIG. 8) and quench the free radicals generated from irradiation of TiO₂ (FIG. 2) thereby increasing the sunscreens safety, stability and ultimate efficacy.

### Example 10

### Stabilization of a 3% Avobenzone Cream

Following the convincing results from the studies described above, a preliminary study with a basic cream containing 3% avobenzone labelled 703-1, to which phytoene and phytofluene (0.0075%), was added and labelled 703-2, was conducted to determine the contribution of phytofluene to sun protection and photo stability of avobenzone under UVA following the Colipa 2009 in vitro Standard Guidelines.

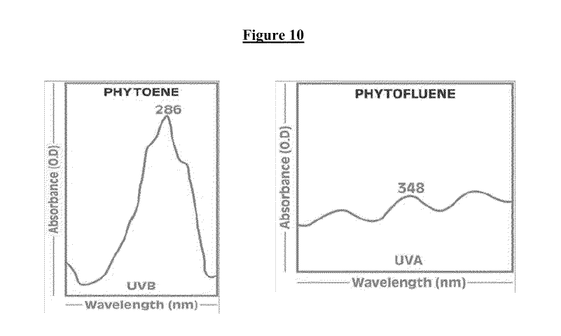

Following the irradiation with UV light as detailed above, the composition retained a most of the avobenzone and showed no signs of chemical or physical degradation. See FIG. 9.

Procedure brief:

Approximately 1.7 mg/cm² of each composition is applied to 5 PMMA plates (Helioplates-HD-6, Helioscreen®, Source: Labsphere, North Sutton, N.H.), respectively, and allowed to equilibrate for at least 15 minutes.

Absorbance spectra from 5 locations on each plate are measured using a UV2000 Sunscreen Analyzer (Labsphere, North Sutton, N.H.). Before and after irradiation with UVA doses of 10 J/cm² administered using an Atlas Suntest® CPS+UV Irradiation System (Atlas Materials Testing, Inc. Chicago) (FIG. 4) which allowed measuring boost to SPF and UVA-PF due to the fact that the energy source of the Suntest® lamp was not strong enough to degrade the avobenzone and only an addition to UVA-Pf with the phytofluene was measured. Plate temperatures are 25-35° C.

Mean UVA protection factors at each UV dose are then computed for each test product, using Colipa UVA Protection Factor Test Method Spreadsheet, and mean absorbance spectra from 290 to 400 nm is obtained. See FIG. 9.

### Example 11

### Stabilization of Sunscreen Compositions by Adding at Least One Additional Carotenoid Other Than Phytoene and Phytofluene

The additional carotenoid is selected from the group consisting of zeta-carotene, beta-carotene, lycopene, zeaxanthin, astaxanthin, cataxanthin, zeaxanthin, lutein, canthaxanthin and the like, and any combination thereof. The ratio between the phytoene and phytofluene mixture to the other carotenoid is from about 1:1 to about 50:1, alternatively from 1:1 to about 1:50. The composition optionally comprises inactive ingredients selected from the group consisting of, but not limited to anti-oxidants, anti-inflammatory agents and vitamins.

### Example 12

### Model Sunscreen Composition in Need of Stabilization

0.5-4% Avobenzone (butyl-methoxydibenzoylmethane)

0-6% Oxybenzone

0-25% Titanium dioxide

0-6% Zinc oxide

Additional sunscreen ingredients q.s.

Humectants (e.g. glycerin, urea, glycols, etc) 1-15%

Emollients (e.g. oils, esters, silicones, etc) 1-25%

Thickening polymer 0.1-2.0%

pH control agents q.s. to pH 4.5-7.5

Additional actives (e.g. vitamins, peptides, etc) q.s.

Preservatives q.s.

Perfume q.s.

Water q.s. to 100%

### Example 13

### Examples of Stabilizing Compositions

Carotenoid compositions based on the instant invention, which may be used for the stabilization of sunscreens are exemplified below:

1. A squalane based composition comprising 1 part of a phytoene or phytofluene or mixtures thereof in 4.3/1 ratio, and 50 parts of lycopene.

2. A squalane based composition comprising 1 part of a phytoene or phytofluene or mixtures thereof in 4.3/1 ratio, and 50 parts of β carotene.

3. A squalane based composition comprising 1 part of phytoene, phytofluene or mixtures thereof wherein phytoene, phytofluene or mixtures thereof are presort: in 4.3/1 ratio, and 50 parts of a carotene and lycopene mixture

4. A squalane based composition comprising 1 part of phytoene, phytofluene or mixtures thereof wherein phytoene, phytofluene or mixtures thereof are present in 4.3/1 ratio, and 50 parts of a β carotene and zeta carotene mixture

5. A squalane based composition comprising 1 part of phytoene, phytofluene or mixtures thereof wherein phytoene, phytofluene or mixtures thereof are present in 4.3/1 ratio, and 50 parts of a lycopene, β carotene and zeta carotene

6. A squalane based composition comprising 50 parts of phytoene, phytofluene or mixtures thereof wherein phytoene, phytofluene or mixtures thereof are present in 4.3/1 ratio, and 1 part of lycopene.

7. A squalane based composition comprising 50 parts of phytoene, phytofluene or mixtures thereof, wherein phytoene, phytofluene or mixtures thereof are present in 4.3/1 ratio, and 1 part of β carotene.

8. A squalane based composition comprising 50 parts of phytoene, phytofluene or mixtures thereof wherein phytoene, phytofluene or mixtures thereof are present in 4.3/1 ratio, and 1 part of a β carotene and lycopene mixture

9. A squalane based composition comprising 50 parts of phytoene, phytofluene or mixtures thereof in 4.3/1 ratio, and 1 part of a β carotene and zeta carotene mixture

10. A squalane based composition comprising 50 parts of phytoene, phytofluene or mixtures thereof in 4.3/1 ratio, and 1 part of a lycopene, β carotene and zeta carotene.

The mixtures of additional carotenoids (lycopene, β-carotene, zeta carotene), to be added to the phytoene or phytofluene or their mixtures are present in ratios between 1:10 and 10:1.

All references, including publications, patent applications, and patents, cited herein are hereby incorporated by reference to the same extent as if each reference were individually and specifically indicated to be incorporated by reference and were set forth in its entirety herein. In addition, the citation or identification of any reference in this application shall not be construed as an admission that such reference qualifies as prior art with respect to the present invention.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A sunscreen composition comprising at least one sunscreen active, wherein said sunscreen active and sunscreen composition are stabilized by the addition of natural or synthetic phytoene or phytofluene or mixtures thereof and optionally at least one natural or synthetic carotenoid other than phytoene/phytofluene.

2. The composition of claim 1, wherein the ratio between phytoene and phytofluene in said mixtures thereof is between 200:1 and 1:200 w/w %.

3. The composition of claim 1, wherein at least one of the sunscreen actives is avobenzone.

4. The composition of claim 1, wherein the at least one of the sunscreen actives is avobenzone and the other optional sunscreen actives are selected from the group comprising Oxybenzone, Sulisobenzone, Dioxybenzone, Octabenzone, p-aminobenzoic acid, Pamidate O, phenylbenzimidazole sulfonic acid, cinoxate, dioxybenzone, oxybenzone, homosalate, menthyl anthranilate, octocrylene, octyl methoxy cinnamate, octyl salicylate, sulisobenzone, trolamine salicylate, Ecamsule, titanium oxide, zinc oxide, 4-methylbenzylidene camphor, Tinosorb M, Tinosorb S, Neo Heliopan AP, Memoryl X L, benzophenone-9, benzophenone-3, benzophenone-12, benzophenone-4, benzophenone-8, Uvinul T 150, Uvinul A Plus, Uvasorb HEB, Parsol SLX, isopentenyl-4-methoxycinnamate or combinations thereof

5. The composition of claim 1, wherein further comprising at least one additional natural or synthetic carotenoid other than phytoene or phytofluene, selected from the group comprising zeta-carotene, beta-carotene, lycopene, zeaxanthin, astaxanthin, cataxanthin, zeaxanthin, lutein, canthaxanthin or mixtures thereof.

6. The composition of claim 1, wherein further comprising jojoba oil, natural or synthetic liquid waxes, liquid triglyceride mixtures, nut, fruit, seed, bean, flower, or cereal oils including but not limited to lanolin, crambe, linseed, flax, castor, tea seed, coconut, cottonseed, rapeseed, kapok seed, corn, sunflower seed, soybean, brazil nut, rice bran, quinoa, babassu, macadamia oils or combinations thereof

7. The composition of claim 1, wherein the ratio between the phytoene or phytofluene or mixtures thereof and the natural or synthetic carotenoid other than phytoene/phytofluene is from about 1:1 to about 50:1 or from 1:1 to about 1:50.

8. The composition of claim 1, wherein the ratio between the phytoene or phytofluene or mixtures thereof to the at least one other natural or synthetic carotenoid is from about 1:1 to about 10:1 or from about 1:1 to about 1:10.

9. The composition of claim 1, wherein the stabilization of the sunscreen active and sunscreen composition prevents degradation of the sunscreen active and the sunscreen composition optionally in the presence of inorganic sunscreens selected from the group comprising ZnO, TiO₂, as a result of sunlight, UV radiation selected from UVA, UVB and UVC, free radical attack or combinations thereof.

10. The composition of claim 1, wherein further comprising additional pharmaceutically or cosmetically acceptable ingredients selected from the group comprising antioxidants, anti-inflammatory agents and vitamins.

11. The composition of claim 1, wherein the addition to the composition of a mixture of phytoene, phytofluene and optionally at least one additional natural or synthetic carotenoid is conducive to an improved SPF and/or UVA-PF and UVA protection of the composition.

12. The composition of claim 1, wherein in the form of a cream, lotion, spray, gel, roll-on, lipstick or other pharmaceutically or cosmetically acceptable topical product.

13. A method for stabilization and preventing the degradation of sunscreen actives and sunscreen compositions by sunlight or UV light, selected from UVA, UVB and UVC and combinations thereof, by addition of phytoene or phytofluene or mixtures thereof and optionally at least one additional natural or synthetic carotenoid other than phytoene/phytofluene.

14. The method of claim 13, wherein the ratio between phytoene and phytofluene in said mixtures thereof is between 200:1 and 1:200 w/w %.

15. The method of claim 13, wherein the ratio between phytoene, phytofluene in said mixtures thereof is between 10:1 and 1:10 w/w %.

16. The method of claim 13, wherein at least one of the sunscreen actives is avobenzone.

17. The method of claim 13, wherein at least one of the sunscreen actives is avobenzone and the other sunscreen actives are selected from the group comprising Oxybenzone, Sulisobenzone, Dioxybenzone, Octabenzone, p-aminobenzoic acid, Pamidate O, phenylbenzimidazole sulfonic acid, cinoxate, dioxybenzone, oxybenzone, homosalate, menthyl anthranilate, octocrylene, octyl methoxy cinnamate, octyl salicylate, sulisobenzone, trolamine salicylate, Ecamsule, titanium oxide, zinc oxide, 4-methylbenzylidene camphor, Tinosorb M, Tinosorb S, Neo Heliopan AP, Memoryl XL, benzophenone-9, benzophenone-3, benzophenone-12, benzophenone-4, benzophenone-8, Uvinul T 150, Uvinul A Plus, Uvasorb HEB, Parsol SLX, isopentenyl-4-methoxycinnamate or combinations thereof.

18. The method of claim 13, wherein further comprising an additional natural or synthetic carotenoid selected from the group comprising zeta-carotene, beta-carotene, lycopene, zeaxanthin, astaxanthin, cataxanthin, zeaxanthin, lutein, canthaxanthin or mixtures thereof.

19. The method of claim 13, wherein the stabilization of the sunscreen active and sunscreen composition prevents degradation of the sunscreen active as a result of sunlight, free radical attack or combinations thereof.

20. The method of claim 13, wherein the stabilization of the sunscreen active and sunscreen composition prevents degradation of the sunscreen active and sunscreen composition in the presence of inorganic sunscreens selected from the group comprising ZnO, TiO₂ as a result of sunlight, free radical attack or combinations thereof.
